(19) United States
(12) Patent Application Publication
(10) Pub. No.: US 2009/0270739 A1
(43) Pub. Date: Oct. 29, 2009

(54) REAL-TIME DETECTION OF VASCULAR CONDITIONS OF A SUBJECT USING ARTERIAL PRESSURE WAVEFORM ANALYSIS

(75) Inventors: Feras Hatib, Irvine, CA (US); Kathryn Willybiro, Huntington Beach, CA (US); Lina Derderian, Trabuco Canyon, CA (US); Luchy Roteliuk, Lake Forest, CA (US)

Correspondence Address: EDWARDS LIFESCIENCES CORPORATION, LEGAL DEPARTMENT, ONE EDWARDS WAY, IRVINE, CA, 92614, US

(73) Assignee: EDWARDS LIFESCIENCES CORPORATION, Irvine, CA (US)

(21) Appl. No.: 12/360,816

(22) Filed: Jan. 27, 2009

Related U.S. Application Data
(60) Provisional application No. 61/024,638, filed on Jan. 30, 2008.

(51) Int. Cl. A61B 5/02

(52) U.S. Cl. 600/485; 600/481

(57) ABSTRACT

Methods for the detection of vascular conditions such as vasodilation in a subject are described. The methods involve receiving a signal corresponding to an arterial blood pressure and calculating one or more cardiovascular parameters from the arterial blood pressure. The cardiovascular parameters are calculated using factors impacted by vascular conditions such as vasodilation. Factors impacted by these vascular conditions include the area under the systolic portion of the arterial blood pressure signal, the duration of systole, and the ratio of the duration of the systole to the duration of the diastole. By monitoring cardiovascular parameters that are calculated using factors impacted by vascular conditions such as vasodilation for changes indicating the vascular conditions, such vascular conditions can be detected.

### CROSS-REFERENCE TO RELATED APPLICATIONS

This application claims the benefit of priority to U.S. Provisional Application No. 61/024,638, filed on Jan. 30, 2008, which is incorporated by reference herein in its entirety.

### BACKGROUND

Arterial blood pressure based methods for the determination of cardiac output (CO) are based on the relationship that exists in the arterial system between pulsatile flow and pulsatile pressure. Most known arterial blood pressure based systems rely on the pulse contour method (PCM), which calculates an estimate of CO from characteristics of the beat-to-beat arterial pressure waveform. In the PCM, “Windkessel” (German for “air chamber”) parameters (characteristic impedance of the aorta, compliance, and total peripheral resistance) are used to construct a linear or non-linear hemodynamic model of the aorta. In essence, blood flow is analogized to a flow of electrical current in a circuit in which an impedance is in series with a parallel-connected resistance and capacitance (compliance). The theoretic pressure that determines stroke volume, i.e., cardiac output, is the proximal aortic pressure. Unfortunately, proximal aortic pressure is not routinely clinically available because the central aortic pressure signal cannot be obtained without complicated clinical procedures involving cardiac catheterization. Clinically, the arterial pressures (e.g., radial, brachial, and femoral) are used instead. The radial artery is the most commonly utilized site because of ease of cannulation and low risk of complications.

Differences in pressure are known to exist within the systemic arterial system mainly as a result of differences in wave reflection. The effect of wave reflection is that the pulse pressure does not have the same amplitude for the central and peripheral arteries, but rather is amplified toward the periphery. In normal hemodynamic conditions, the arterial pulse pressure is higher in the peripheral arteries than in the aorta. This phenomenon of increased arterial pressure amplitude is well established and peripheral pressure is routinely used with correction factors in calculations of cardiac output.

### SUMMARY

Methods for the detection of a vascular condition in a subject are described. The vascular condition includes different cardiovascular hemodynamic conditions and states, such as, for example, vasodilation, vasoconstriction, peripheral pressure/flow decoupling, conditions where the peripheral arterial pressure is not proportional to the central aortic pressure, and conditions where the peripheral arterial pressure is lower than the central aortic pressure. One method of detecting a vascular condition in a subject involves receiving a signal corresponding to an arterial blood pressure and calculating a cardiovascular parameter from the arterial blood pressure. The cardiovascular parameter is calculated based on a set of factors including one or more parameters effected by the vascular condition. Examples of parameters effected by the vascular condition include (a) a parameter based on the area under the systolic portion of the arterial blood pressure signal, (b) a parameter based on the duration of systole, and (c) a parameter based on the ratio of the duration of the systole to the duration of the diastole. Additional parameters can be used in calculating the cardiovascular parameter including one or more of (d) a parameter based on the shape of the arterial blood pressure signal and at least one statistical moment of the arterial blood pressure signal having an order of one or greater, (e) a parameter corresponding to the heart rate, and (f) a set of anthropometric parameters of the subject. The cardiovascular parameter is then monitored for a statistically significant change over time with the detection of a statistically significant change in the cardiovascular parameter indicating the vascular condition.

Further methods of detecting a vascular condition in a subject involve receiving a signal corresponding to an arterial blood pressure and calculating a first cardiovascular parameter and a second cardiovascular parameter from the arterial blood pressure. The first cardiovascular parameter is calculated based on a first set of factors including one or more of (a) a parameter based on the shape of the beat-to-beat arterial blood pressure signal and at least one statistical moment of the arterial blood pressure signal having an order of one or greater, (b) a parameter based on a heart rate of the subject, and (c) a set of anthropomorphic parameters of the subject. The second cardiovascular parameter is calculated based on a second set of factors including one or more parameters effected by the vascular parameter. Examples of parameters effected by the vascular parameter include (a) a parameter based on the area under the systolic portion of the arterial blood pressure signal, (b) a parameter based on the duration of systole, and (c) a parameter based on the ratio of the duration of the systole to the duration of the diastole. Finally, the first cardiovascular parameter is subtracted from the second cardiovascular parameter to create a difference factor or a ratio between the second cardiac parameter and the first cardiovascular parameter is determined. A difference factor of greater than a predetermined threshold value or a ratio greater than a predetermined value indicates the vascular condition.

### DESCRIPTION OF DRAWINGS

FIG. 1 shows simultaneously recorded pressure waveforms in the ascending aorta (Aortic), femoral artery (Femoral), and radial artery (Radial) in a porcine animal model during normal hemodynamic conditions.

FIG. 2 shows simultaneously recorded pressure waveforms in the ascending aorta (Aortic), femoral artery (Femoral), and radial artery (Radial) in a porcine animal model during Endotoxin shock (septic shock) resuscitated with large amounts of fluids and vasopressors.

FIG. 3 shows an example of a complex blood pressure curve over one beat-to-beat heart cycle.

FIG. 4 shows a discrete-time representation of the pressure waveform of FIG. 3.

FIG. 5 shows the area under the systolic portion of the arterial pressure waveform.

FIG. 6 shows the statistical distributions of the area under the systolic phase of the arterial pressure waveform for normal subjects and hyperdynamic subjects.

FIG. 7 shows the duration of the systole for an arterial pressure waveform.

FIG. 8 shows the statistical distribution of the duration of the systole of the arterial pressure waveform for normal subjects and hyperdynamic subjects.

FIG. 9 shows the duration of the systole and the duration of the diastole for an arterial pressure waveform.

FIG. 10 is the statistical distribution of the duration of the diastolic phase for high heart rate subjects in normal hemodynamic conditions (dashed line) and hyperdynamic conditions (thick line)—the distribution for all the patients combined is also shown (thin line).

FIG. 11 is the statistical distribution of the duration of the systolic phase for high heart rate subjects in normal hemodynamic conditions (dashed line) and hyperdynamic conditions (thick line)—the distribution for all the patients combined is also shown (thin line).

FIG. 12 is a graph showing a calculation of χ (thin black line), χₕ (grey line) and the gold standard arterial tone (thick black line) over time for a subject entering hyperdynamic conditions.

FIG. 13 is a block diagram showing the main components of a system to implement the methods described herein.

### DETAILED DESCRIPTION

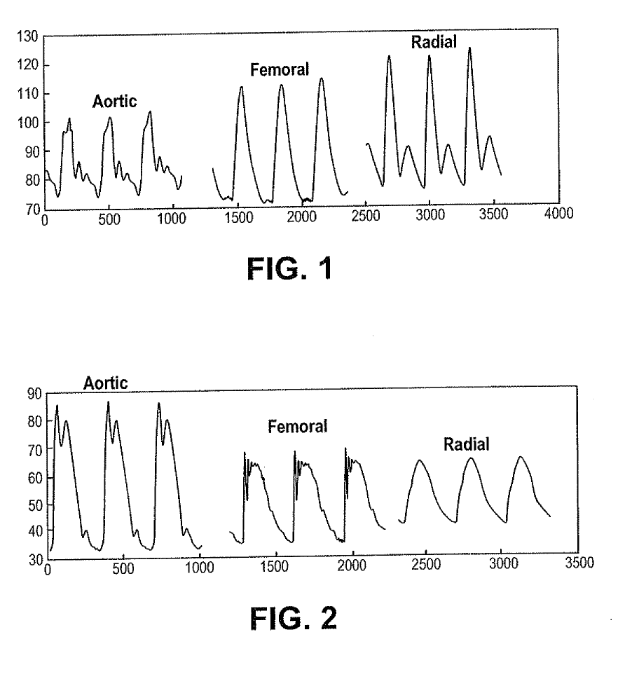

Methods for the detection of a vascular condition in a subject are described. The vascular condition may include different cardiovascular hemodynamic conditions and states, such as, for example, vasodilation, vasoconstriction, peripheral pressure/flow decoupling, conditions where the peripheral arterial pressure is not proportional to the central aortic pressure, and conditions where the peripheral arterial pressure is lower than the central aortic pressure. As used herein, the phrase vasodilation means a condition in which the arterial and peripheral arterial pressure and flow are decoupled from the central aortic pressure and flow, and the term peripheral arteries is intended to mean arteries located away from the heart, e.g., radial, femoral, or brachial arteries. Decoupled arterial pressure means that the normal relationship between arterial, peripheral arterial, and central pressure is not valid and the arterial and peripheral arterial pressure can not be used to determine the central arterial pressure. This also includes conditions in which the peripheral arterial pressure is not proportional or is not a function of the central aortic pressure. Under normal hemodynamic conditions, blood pressure increases the further away from the heart the measurement is taken. Such a pressure increase if shown in FIG. 1, i.e., the amplitude of a pressure wave measured at radial arteries is greater than the pressure measured at the femoral artery, which in turn is greater than the aortic pressure. These differences in pressure are related to wave reflection, i.e., pressure is amplified toward the periphery.

This normal hemodynamic relationship of pressures, i.e., an increase in pressure away from the heart, is often relied upon in medical diagnosis. However, under hyperdynamic conditions, this relationship can become inverted with the arterial pressure becoming lower than the central aortic pressure. This reversal has been attributed, for example, to arterial tone in the peripheral vessels, which is suggested to impact the wave reflections discussed above. Such a hyperdynamic condition is shown in FIG. 2, i.e., the amplitude of a pressure wave measured at radial arteries is lower than the pressure measured as the femoral artery, which in turn is lower than the aortic pressure. Drugs that dilate small peripheral arteries (e.g., nitrates, ACE inhibitors, and calcium inhibitors) are thought to contribute to hyperdynamic conditions. These types of severe vasodilatory conditions are also often observed in situations right after cardiopulmonary bypass (coronary bypass), in which the radial arterial pressure underestimates the pressure in the aorta. Substantial central to peripheral pressure differences, where the peripheral arterial pressure underestimates the central aortic pressure, are usually observed in patients with severe sepsis who are treated with large amount of fluids and high-dose vasopressors, leading to severe vasodilation. Very similar conditions are also observed in patient with end stage liver disease. As will be well appreciated by those of skill in the art, certain treatments for subjects in normal hemodynamic conditions will be approached differently than for subjects in hyperdynamic conditions. Thus, the presently disclosed methods for detecting vascular conditions such as vasodilation in a subject will be very useful to those of skill in the art.

In general, these methods involve monitoring a cardiovascular parameter that implicates a vascular condition in a subject to detect a change that indicates the vascular condition. An example of such a change is a statistically significant change in the cardiovascular parameter, such as a change of greater than one standard deviation. Another example of a change that indicates a vascular condition is a difference between a cardiovascular parameter impacted by hyperdynamic conditions and a cardiovascular parameter not impacted by hyperdynamic conditions of greater than a predetermined threshold. A further example of a change that indicates a vasodilatory condition is a ratio between a cardiovascular factor impacted by hyperdynamic conditions and a cardiovascular parameter not impacted by hyperdynamic conditions that is greater than a predetermined value. These cardiovascular parameters are calculated and the above listed changes are monitored continuously as a subject's arterial blood pressure is monitored. The cardiovascular parameter can be, for example, arterial compliance, arterial elasticity, peripheral resistance, arterial tone, arterial flow, stroke volume, or cardiac output. The detection of vascular conditions such as vasodilation in a subject indicates, for example, the occurrence of hyperdynamic cardiovascular conditions, hyperdynamic decoupling of the arterial pressure from the central aortic pressure, that the arterial pressure is lower than the central aortic pressure, or that the arterial pressure is not proportional to the central aortic pressure.

More specifically, a method of detecting a vascular condition in a subject involves receiving a signal corresponding to an arterial blood pressure and calculating a cardiovascular parameter from the arterial blood pressure. The cardiovascular parameter is calculated based on a set of factors including one or more parameters effected by the vascular condition. Examples of parameters effected by the vascular condition include (a) a parameter based on the area under the systolic portion of the arterial blood pressure signal, (b) a parameter based on the duration of systole, and (c) a parameter based on the ratio of the duration of the systole to the duration of the diastole. The factors used to calculate the cardiovascular parameter can further include one or more of (d) a parameter based on the shape of the arterial blood pressure signal and at least one statistical moment of the arterial blood pressure signal having an order of one or greater, (e) a parameter corresponding to the heart rate, and (f) a set of anthropometric parameters of the subject. The cardiovascular parameter is then monitored for a statistically significant change over time with the detection of a statistically significant change in the cardiovascular parameter indicating the vascular condition. The statistically significant change is, for example, a change of greater than one standard deviation or a change of greater than one standard deviation of a parameter when compared to the distribution of the parameter in normal subjects not experiencing the vascular condition.

Another method of detecting a vascular condition in a subject involves receiving a signal corresponding to an arterial blood pressure and calculating a first cardiovascular parameter and a second cardiovascular parameter from the arterial blood pressure. The first cardiovascular parameter is calculated based on a first set of factors including one or more of (a) a parameter based on the shape of the beat-to-beat arterial blood pressure signal and at least one statistical moment of the arterial blood pressure signal having an order of one or greater, (b) a parameter based on a heart rate of the subject, and (c) a set of anthropomorphic parameters of the subject. The second cardiovascular parameter is calculated based on a second set of factors including one or more parameters effected by the vascular condition. Examples of parameters effected by the vascular condition include (a) a parameter based on the area under the systolic portion of the arterial blood pressure signal, (b) a parameter based on the duration of systole, and (c) a parameter based on the ratio of the duration of the systole to the duration of the diastole. Finally, the first cardiovascular parameter is subtracted from the second cardiovascular parameter to create a difference factor. A difference factor of greater than a predetermined threshold value indicates the vascular condition. The predetermined value can represent a statistically significant change in the difference factor over time, e.g., a change of greater than one standard deviation of a parameter when compared to the distribution of the parameter in normal subjects not experiencing the vascular condition. Examples of predetermined threshold values include 1.5 L/minute or greater, 1.6 L/minute or greater, 1.7 L/minute or greater, 1.8 L/minute or greater, 1.9 L/minute or greater, 2 L/minute or greater, 2.1 L/minute or greater, 2.2 L/minute or greater, 2.3 L/minute or greater, 2.4 L/minute or greater, and 2.5 L/minute or greater.

A further method of detecting a vascular condition in a subject involves receiving a signal corresponding to an arterial blood pressure and calculating a first cardiovascular parameter and a second cardiovascular parameter from the arterial blood pressure. The first cardiovascular parameter is calculated based on a first set of factors including one or more of (a) a parameter based on the shape of the beat-to-beat arterial blood pressure signal and at least one statistical moment of the arterial blood pressure signal having an order of one or greater, (b) a parameter based on a heart rate of the subject, and (c) a set of anthropomorphic parameters of the subject. The second cardiovascular parameter is calculated based on a second set of factors including one or more parameters effected by the vascular condition. Examples of parameters effected by the vascular condition include (a) a parameter based on the area under the systolic portion of the arterial blood pressure signal, (b) a parameter based on the duration of systole, and (c) a parameter based on the ratio of the duration of the systole to the duration of the diastole. A ratio of the second cardiovascular parameter to the first cardiovascular parameter of greater than a predetermined value indicates the vascular condition. Examples of predetermined values include 1.1 or greater, 1.2 or greater, 1.3 or greater, 1.4 or greater, 1.5 or greater, 1.6 or greater, 1.7 or greater, 1.8 or greater, 1.9 or greater, and 2.0 or greater.

The cardiovascular parameters used in the methods described herein are calculated from signals based on arterial blood pressure or signals proportional to arterial blood pressure. The calculation of cardiovascular parameters, such as arterial compliance (arterial tone), is described in U.S. patent application Ser. No. 10/890,887, filed Jul. 14, 2004, which is incorporated herein by reference in its entirety. The factors and data used in calculating the cardiovascular parameters for use with the methods disclosed herein, including the parameters discussed in U.S. patent application Ser. No. 10/890,887, are described below.

### Pressure Waveforms

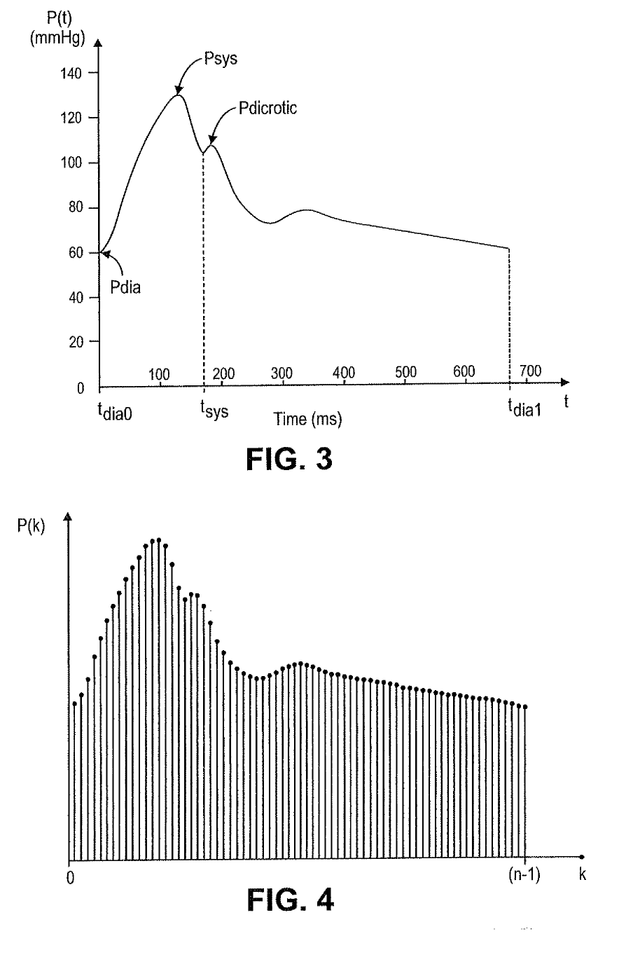

FIG. 3 is an example of an arterial pressure waveform, P(t), taken over a single heart cycle. This heart cycle starts at the point of diastolic pressure P_{dia} at time t_{dia0}, through the time t_{sys} of up to systolic pressure P_{sys}, to a time t_{dia1} at which the blood pressure once again reaches P_{dia}.

Signals useful with the present methods include cardiovascular parameters based on arterial blood pressure or any signal that is proportional to arterial blood pressure, measured at any point in the arterial tree, e.g., radial, femoral, or brachial, either invasively or non-invasively. If invasive instruments are used, in particular, catheter-mounted pressure transducers, then any artery is a possible measurement point. Placement of non-invasive transducers will typically be dictated by the instruments themselves, e.g., finger cuffs, upper arm pressure cuffs, and earlobe clamps. Regardless of the specific instrument used, the data obtained will ultimately yield an electric signal corresponding (for example, proportional) to arterial blood pressure.

As illustrated in FIG. 4, analog signals such as arterial blood pressure can be digitized into a sequence of digital values using any standard analog-to-digital converter (ADC). In other words, arterial blood pressure, t0≦t≦tf, can be converted, using known methods and circuitry, into the digital form P(k), k=0, (n−1), where t0 and tf are initial and final times of the measurement interval and n is the number of samples of arterial blood pressure to be included in the calculations, distributed usually evenly over the measurement interval.

### Moments

Now consider an ordered collection of m values, that is, a sequence Y(i), where i=1, . . . (m−1). As is well known from the field of statistics, the first four moments μ₁, μ₂, μ₃, and μ₄ of Y(i) can be calculated using known formulas, where μ₁ is the mean (i.e., arithmetic average), μ₂=σ² is the variation (i.e., the square of the standard deviation σ), μ₃ is the skewness, and μ₄ is the kurtosis. Thus:

μ₁Y_{avg}=1/m*Σ(Y(i)) (Formula 1)

μ₂=σ²=1/(m−1)*Σ(Y(i)−Y_{avg})² (Formula 2)

μ₃=1/(m−1)*Σ[(Y(i)−Y_{avg})/σ]³ (Formula 3)

μ₄=σ/(m−1)*Σ[(Y(i)−Y_{avg})/σ]⁴ (Formula 4)

In general, the β-th moment μₚ can be expressed as:

μ_{β}=1(m−1)*1/σ^{β}*Σ[(Y)(i)−Y_{avg})]^{β} (Formula 5)

where i=0, . . . , (m−1). The discrete-value formulas for the second through fourth moments usually scale by 1/(m−1) instead of 1/m for well-known statistical reasons.

The methods described herein utilize a compliance factor or an arterial tone factor that is a function not only of the four moments of the pressure waveform P(k), but also of a pressure-weighted time vector. Standard deviation σ provides one level of shape information in that the greater σ is, the more “spread out” the function Y(i) is, i.e., the more it tends to deviate from the mean. Although the standard deviation provides some shape information, its shortcoming can be easily understood by considering the following: the mean and standard deviation will not change if the order in which the values making up the sequence Y(i) is “reversed,” that is, Y(i) is reflected about the i=0 axis and shifted so that the value Y(m−1) becomes the first value in time.

Skewness is a measure of lack of symmetry and indicates whether the left or right side of the function Y(i), relative to the statistical mode, is heavier than the other. A positively skewed function rises rapidly, reaches its peak, then falls slowly. The opposite would be true for a negatively skewed function. The point is that the skewness value includes shape information not found in the mean or standard deviation values—in particular, it indicates how rapidly the function initially rises to its peak and then how slowly it decays. Two different functions may have the same mean and standard deviation, but they will then only rarely have the same skewness.

Kurtosis is a measure of whether the function Y(i) is more peaked or flatter than a normal distribution. Thus, a high kurtosis value will indicate a distinct peak near the mean, with a drop thereafter, followed by a heavy “tail.” A low kurtosis value will tend to indicate that the function is relatively flat in the region of its peak. A normal distribution has a kurtosis of 3.0; actual kurtosis values are therefore often adjusted by 3.0 so that the values are instead relative to the origin.

An advantage of using the four statistical moments of the beat-to-beat arterial pressure waveform is that the moments are accurate and sensitive mathematical measures of the shape of the beat-to-beat arterial pressure waveform. As arterial compliance and peripheral resistance directly affect the shape of the arterial pressure waveform, the effect of arterial compliance and peripheral resistance could be directly assessed by measuring the shape of the beat-to-beat arterial pressure waveform. The shape sensitive statistical moments of the beat-to-beat arterial pressure waveform along with other arterial pressure parameters described herein could be effectively used to measure the combined effect of vascular compliance and peripheral resistance, i.e., the arterial tone. The arterial tone represents the combined effect of arterial compliance and peripheral resistance and corresponds to the impedance of the well known 2-element electrical analog equivalent model of the Windkessel hemodynamic model, consisting of a capacitive and a resistive component. By measuring arterial tone, several other parameters that are based on arterial tone, such as arterial elasticity, stroke volume, and cardiac output, also could be directly measured. Any of those parameters could be used to detect vascular conditions such as, for example, vasodilation, vasoconstriction, or peripheral pressure decoupling.

### Pressure Waveform Moments

When the first four moments μ_{1P}, μ_{2P}, μ_{3P}, and μ_{4P} of the pressure waveform P(k) are calculated and used in the computation of the arterial tone factor, where μ_{1P} is the mean, μ_{2P} P=σ_{P}² is the variation, that is, the square of the standard deviation σ_{P}; μ_{3P} is the skewness, and μ_{4P} is the kurtosis, where all of these moments are based on the pressure waveform P(k). Formulas 1-4 above may be used to calculate these values after substituting P for Y, k for i, and n for m.

Formula 2 above provides the “textbook” method for computing a standard deviation. Other, more approximate methods may also be used. For example, at least in the context of blood pressure-based measurements, a rough approximation to ν_{P} is to divide by three the difference between the maximum and minimum measured pressure values, and that the maximum or absolute value of the minimum of the first derivative of the P(t) with respect to time is generally proportional to σ_{P}.

### Pressure-Weighted Time Moments

As FIG. 4 illustrates, at each discrete time k, the corresponding measured pressure will be P(k). The values k and P(k) can be formed into a sequence T(j) that corresponds to a histogram, meaning that each P(k) value is used as a “count” of the corresponding k value. By way of a greatly simplified example, assume that the entire pressure waveform consists of only four measured values P(1)=25, P(2)=50, P(3)=55, and P(4)=35. This could then be represented as a sequence T(j) with 25 ones, 50 twos, 55 threes, and 35 fours:

T(j)=1,1, . . . , 1,2,2, . . . , 2,3,3, . . . , 3,4,4, . . . , 4

This sequence would thus have 25+50+55+35=165 terms.

Moments may be computed for this sequence just as for any other. For example, the mean (first moment) is:

μ_{1T}=(1*25+2*50+3*55+4*35)/165=430/165=2.606 (Formula 6)

and the standard deviation σ_{T} is the square root of the variation μ_{2T}:

SQRT[1/164*25(1-2.61)²+50(2-2.61)²+55(3-2.61)²+35(4-2.61)²]=0.985

The skewness μ_{3T} and kurtosis μ_{4T} can be computed by similar substitutions in Formulas 3 and 4:

μ_{3T}={1/(164)*(1/σ_{T}³)Σ[P(k)*(k−μ_{1T})³]} (Formula 7)

μ_{4T}={1/(164)*(1/σ_{T}⁴)Σ[P(k)*(k−μ_{1T})⁴]} (Formula 8)

where k=1, . . . , (m−1).

As these formulas indicate, this process in effect “weights” each discrete time value k by its corresponding pressure value P(k) before calculating the moments of time. The sequence T(j) has the very useful property that it robustly characterizes the timing distribution of the pressure waveform. Reversing the order of the pressure values P(k) will in almost all cases cause even the mean of T(j) to change, as well as all of the higher-order moments. Moreover, the secondary “hump” that normally occurs at the dicrotic pressure P_{dicrotic} also noticeably affects the value of kurtosis μ_{4T}; in contrast, simply identifying the dicrotic notch in the prior art, such as in the Romano method, requires noisy calculation of at least one derivative.

The pressure weighted moments provide another level of shape information for the beat-to-beat arterial pressure signal, as they are very accurate measures of both the amplitude and the time information of the beat-to-beat-arterial pressure signal. Use of the pressure weighted moments in addition to the pressure waveform moments can increase the accuracy of the of arterial tone determination.

### Parameter Set

One cardiovascular parameter useful with the methods described herein is the arterial tone factor K, which can be used as a cardiovascular parameter by itself or in the calculation of other cardiovascular parameters such as stroke volume or cardiac output. Calculation of the arterial tone K uses all four of the pressure waveform and pressure-weighted time moments. Additional values are included in the computation to take other known characteristics into account, e.g., patient-specific complex pattern of vascular branching. Examples of additional values include, heart rate HR (or period of R-waves), body surface area BSA, or other anthropometric parameters of the subject, a compliance value C(P) calculated using a known method such as described by Langwouters, which computes compliance as a polynomial function of the pressure waveform and the patient's age and sex, a parameter based on the shape of the arterial blood pressure signal and at least one statistical moment of the arterial blood pressure signal having an order of one or greater, a parameter based on the area under the systolic portion of the arterial blood pressure signal, a parameter based on the duration of the systole, and a parameter based on the ratio of the duration of the systole to the duration of the diastole.

These last three cardiovascular parameters, i.e., the area under the systolic portion of the arterial blood pressure signal, the duration of the systole, and the ratio of the duration of the systole to the duration of the diastole, are impacted by arterial tone and vascular compliance and, thus, vary between subjects in normal hemodynamic conditions and subjects in hyperdynamic conditions. Because these three cardiovascular parameters vary between normal and hyperdynamic subjects the methods described herein can use these cardiovascular parameters to detect vasodilation or vasoconstriction in the peripheral arteries of a subject.

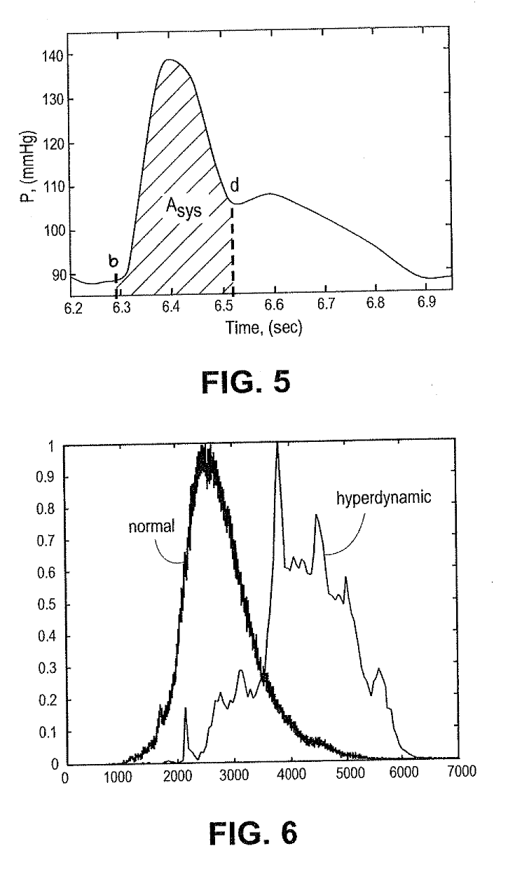

The area under the systolic portion of an arterial pressure waveform (A_{sys}) is shown graphically in FIG. 5. The area under the systolic portion of the arterial pressure waveform in an arterial pressure signal is defined as the area under the portion of the waveform starting from the beginning of the beat and ending in the dichrotic notch (from point b to point d on FIG. 5). The area under the systole represents the energy of the arterial pressure signal during systole, which is directly proportional to stroke volume and inversely proportional to arterial compliance. When measured over groups of normal and hyperdynamic patients a shift in A_{sys} can be detected. As shown in FIG. 6, the energy of the arterial pressure signal during systole is higher in some subjects in hyperdynamic conditions. Those subjects with higher A_{sys}, are typically subjects with high cardiac output (CO) and low or normal HR, where the elevated CO is mainly caused by elevated heart contractility, which means that those subjects have increased stroke volume and decreased arterial compliance, which is directly reflected in the energy of the arterial pressure signal during systole. The reflected waves, which are usually very intense during many hyperdynamic conditions, may have also significant contribution to the increased energy of the signal during systole.

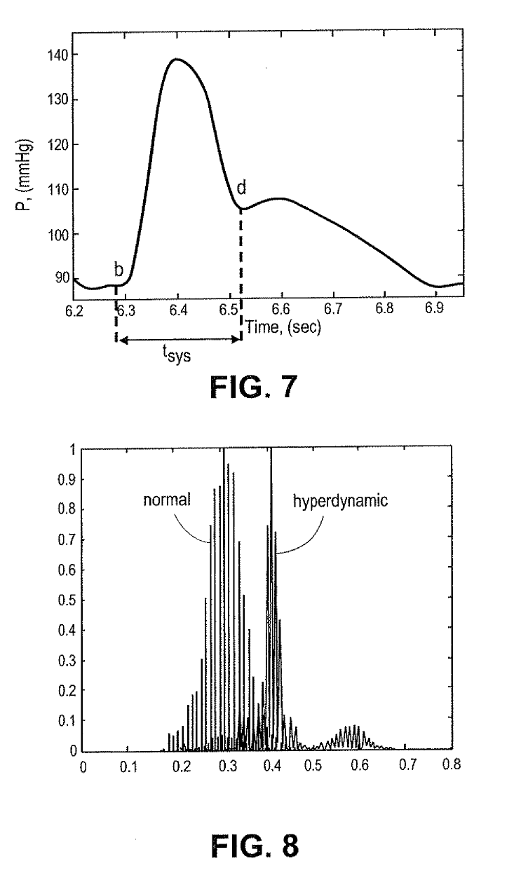

The duration of the systole (t_{sys}) is shown graphically in FIG. 7. The duration of the systole in an arterial pressure waveform is defined as the time duration from the beginning of the beat to the dichrotic notch (from point b to point d on FIG. 7). The duration of the systole is directly affected by the arterial compliance and is relatively independent of the changes in peripheral arterial tone, except when large reflect waves are present. As shown on FIG. 8, the duration of the systole in some hyperdynamic subjects is higher than the duration of the systole in normal subjects (data shifted toward higher t_{sys} values). As seen for the systolic energy, the duration of the systole is typically higher in patients with high CO who also have low or normal HR, where the elevated CO is mainly caused by elevated heart contractility and where the contractility may not have been high enough to increase the systolic energy. The increased stroke volume in those patients is partially due to increased contractility and partially due to increased duration of the systole. Reflected waves play a role here as well.

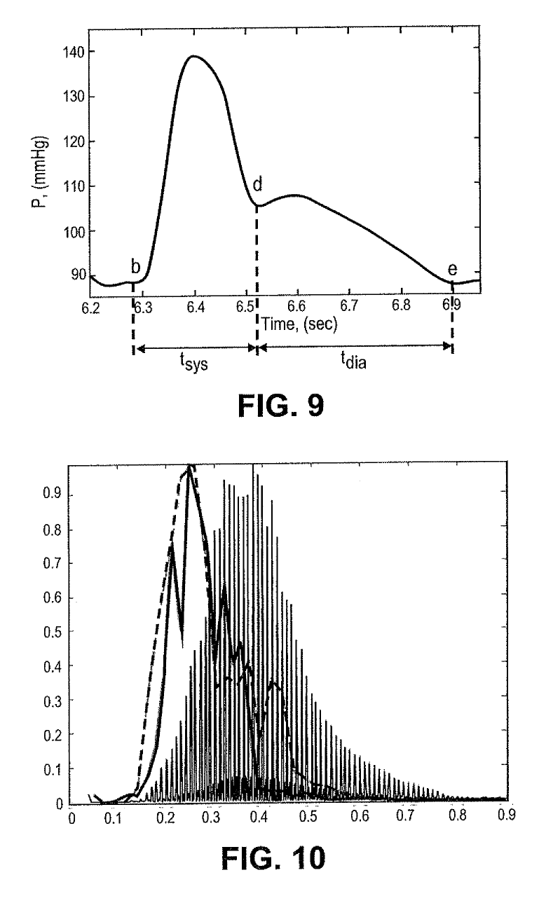

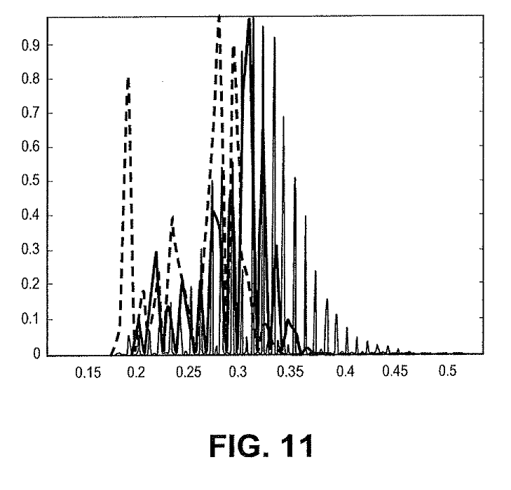

A further parameter that varies between normal and hyperdynamic subjects is the ratio of the duration of the systole (t_{sys}) and the duration of the diastole (t_{dia}), as shown graphically in FIG. 9. The duration of the diastole in an arterial pressure waveform is defined as the time duration from the dichrotic notch to the end of the cardiac cycle (from point d to point e on FIG. 9). In some hyperdynamic conditions, the ratio of the durations of the systole and diastole is significantly higher than that observed in normal hemodynamic conditions. This is typically observed in septic shock patients with elevated CO where HR is also high. In these types of conditions, the systole takes over almost the entire cardiac cycle leaving very little time for the diastole before the next cardiac cycle begins. This is shown in FIGS. 10 and 11, which show the duration of diastole (FIG. 10) and the duration of systole (FIG. 11) during high HR conditions in septic shock patients and in normal patients. As shown in the figures, high HR patients in normal hemodynamic conditions (dashed line) tend to have low durations of both the systole and the diastole, while high HR patients in septic shock (thick line) tend to have low duration of the diastole but normal or high duration of the systole.

### Multivariate Models

In principle, each of these parameters could be monitored individually to detect hyperdynamic conditions. However, such changes are complex and a multivariate model can often provide a more accurate indication. For example, a compliance or arterial tone factor K can be calculated using a set of parameters including one or more of the area under the systolic portion of the arterial blood pressure signal, the duration of the systole, and the ratio of the duration of the systole to the duration of the diastole.

Determining a cardiovascular parameter using an empirical multivariable statistical model involves several steps. First, an approximating function relating a set of clinically derived reference measurements to the cardiovascular parameter is determined. The set of clinically determined reference measurements of the cardiovascular parameter represents clinical measurements of the cardiovascular parameter, e.g., arterial tone, from both subjects not experiencing the vascular condition and subjects experiencing the vascular condition. The approximating function is a function of one or more of (a) a parameter based on the area under the systolic portion of the arterial blood pressure signal, (b) a parameter based on the duration of systole, (c) a parameter based on the ratio of the duration of the systole to the duration of the diastole, and (d) a parameter based on the shape of the arterial blood pressure signal and at least one statistical moment of the arterial blood pressure signal having an order of one or greater. Next a set of arterial blood pressure parameters from the arterial blood pressure signal is determined. The set of arterial blood pressure parameters includes one or more of (a) a parameter based on the area under the systolic portion of the arterial blood pressure signal, (b) a parameter based on the duration of systole, (c) a parameter based on the ratio of the duration of the systole to the duration of the diastole, and (d) a parameter based on the shape of the arterial blood pressure signal and at least one statistical moment of the arterial blood pressure signal having an order of one or greater. Finally, the cardiovascular parameter is estimated by evaluating the approximating function with the set of arterial blood pressure parameters. The set of arterial blood pressure parameters derived from subjects experiencing the vascular condition can optionally be given more weight in the model than the data derived from subjects not experiencing the vascular condition.

An example of a multivariate model to determine an arterial factor impacted by a vascular condition such as vasodilation to be used as a cardiovascular parameter in the methods described herein, involves the use of the following multivariate model (the hyperdynamic model), which uses many of the parameters discussed above and includes the area under systole (A_{sys}), the duration of systole (t_{sys}) and the duration of the diastole (t_{dia}):

Kₕ=χₕ(A_{sys},t_{sys},t_{dia},μ_{T1},μ_{T2}, . . . μ_{Tk},μ_{P1},μ_{P2}, . . . μ_{Pk},C(P),BSA,Age,G . . . ) (Formula 9)

where:
Kₕ is arterial tone of the hyperdynamic model;
χₕ is a multi-regression statistical model;
A_{sys} is the area under systole;
t_{sys} is the duration of the systole;
t_{dia} is the duration of the diastole;
μ_{1T} . . . μ_{kT} are the 1-st to k-th order time domain statistical moments of the arterial pulse pressure waveform (as defined in U.S. patent application Ser. No. 10/890,887, filed Jul. 14, 2004);
μ_{1P} . . . μ_{kP} are the 1-st to k-th order pressure weighted statistical moments of the arterial pulse pressure waveform (as defined in U.S. patent application Ser. No. 10/890,887, filed Jul. 14, 2004);
C(P) is a pressure dependent vascular compliance computed using methods proposed by Langwouters et al 1984 (“The Static Elastic Properties of 45 Human Thoracic and 20 Abdominal Aortas in vitro and the Parameters of a New Model,” J. Biomechanics, Vol. 17, No. 6, pp. 425-435, 1984);
BSA is a patient's body surface area (function of height and weight);
Age is a patient's age; and
G is a patient's gender.

To increase the accuracy of the calculations, the predictor variables set for the multivariate model χₕ are related to the “true” vascular tone measurement (determined as a function of CO measured through thermodilution and the arterial pulse pressure) for a population of test or reference subjects that includes subjects in normal hemodynamic conditions, i.e., not experiencing the vascular condition, and subjects in hyperdynamic conditions, i.e., experiencing the vascular condition, e.g., low arterial tone and marked peripheral decoupling of arterial pressure and flow. Additionally, to further highlight the change from normal hemodynamic conditions to hyperdynamic conditions, the model χₕ is statistically weighted with the data from the hyperdynamic subjects, i.e., the data from the hyperdynamic subjects is weighted more heavily in the model than the data from the normal subjects. The multivariate approximating function is then computed, using known numerical methods, that best relates the parameters of χₕ to a given suite of CO measurements in a predefined manner and weighted on the hyperdynamic side. A polynomial multivariate fitting function is used to generate the coefficients of the polynomial that gives a value of χₕ for each set of the predictor variables. Thus, such a multivariate model has the following general form:

$${χ}_{h} = [ {A}_{h 1} {A}_{h 2} … {A}_{hn } ] * [ \begin{matrix}\begin{matrix}\begin{matrix}{X}_{h 1} \\ {X}_{h 2}\end{matrix} \\ …\end{matrix} \\ {X}_{h n}\end{matrix} ] \qquad ( Formula 10 )$$

where Aₕ₁ . . . . Aₕₙ are the coefficients of the polynomial multi-regression model, and Xₕ, are the model's predictor variables:

$${X}_{n , h 1} = {\prod }_{m} ( {[ \begin{matrix}\begin{matrix}{A}_{s} , {t}_{s} , {t}_{d} , {μ}_{T 1 } … \\ {μ}_{Tk} {μ}_{P 1} … {μ}_{P 1} …\end{matrix} \\ {μ}_{Tk} C ( P ) BSA Age G …\end{matrix} ]}^{\hat{} [ \begin{matrix}{P}_{1 , 1} & … & {P}_{1 , m} \\ … & … & … \\ {P}_{n , 1} & … & {P}_{n , m}\end{matrix} ]} ) \qquad ( Formula 11 )$$

To determine an arterial tone factor to be used as a cardiovascular parameter that does not take into account the parameters identified above that are not impacted by peripheral decoupling, a multivariate model also is used (the normal hemodynamic model) that involves several steps. First an approximating function relating a set of clinically derived reference measurements to the cardiovascular parameter, e.g., arterial tone, is determined. The set of clinically determined reference measurements of the cardiovascular parameter represents clinical measurements of the cardiovascular parameter from subjects not experiencing the vascular condition. The approximating function is a function of one or more of (a) a parameter based on the shape of the arterial blood pressure signal including calculating at least one statistical moment of the arterial blood pressure signal having an order of one or higher, (b) a parameter based on the heart rate, and (c) a set of anthropometric parameters of the subject. Next a set of arterial blood pressure parameters from the arterial blood pressure signal is determined. The set of arterial blood pressure parameters includes one or more of the shape of the arterial blood pressure signal and at least one statistical moment of the arterial blood pressure signal having an order of one or greater, and the heart rate. Next a set of anthropometric parameters of the subject is determined. Finally, the cardiovascular parameter is estimated by evaluating the approximating function with the set of arterial blood pressure parameters and the set of anthropometric parameters of the subject.

An example of such a multivariate model to determine an arterial tone factor not impacted by the vascular condition to be used as a cardiovascular parameter in the methods described herein, involves the use of many of the parameters discussed above but excludes the area under systole (A_{sys}), the duration of systole (t_{sys}) and the duration of the diastole (t_{dia}), i.e., those parameters impacted by the vascular condition:

K=χ(μ_{T1},μ_{T2}, . . . μ_{Tk},μ_{P1},μ_{P2}, . . . μ_{Pk},C(P),BSA,Age,G . . . ) (Formula 12)

Where the parameters K, χ, μ_{1T}, . . . μ_{kT}, μ_{1P} . . . μ_{kP}, C(P), BSA, Age, and G are the same as described above for the hyperdynamic model.

Similar to that discussed above, the predictor variables set for computing the vascular tone factor K, using the multivariate model χ, is related to the “true” vascular tone measurement, determined as a function of CO measured through thermo-dilution and the arterial pulse pressure, for a population of test or reference subjects. This creates a suite of vascular tone measurements, each of which is a function of the component parameters of χ. A multivariate approximating function is then computed, using known numerical methods, that best relates the parameters of χ to a given suite of CO measurements in a predefined manner. A polynomial multivariate fitting function is used to generate the coefficients of the polynomial that gives a value of χ for each set of the predictor variables. Thus, such a multivariate model has the following general form:

$$χ = [ {A}_{1} {A}_{2} … {A}_{n } ] * [ \begin{matrix}\begin{matrix}\begin{matrix}{X}_{1} \\ {X}_{2}\end{matrix} \\ …\end{matrix} \\ {X}_{n}\end{matrix} ] \qquad ( Formula 13 )$$

where A₁ . . . Aₙ, are the coefficients of the polynomial multi-regression model, and χ are the model's predictor variables:

$${X}_{n , 1} = {\prod }_{m} ( {[ \begin{matrix}\begin{matrix}{μ}_{T 1 } … {μ}_{Tk} \\ {μ}_{P 1} … {μ}_{P 1} …\end{matrix} \\ {μ}_{Tk} C ( P ) BSA Age G …\end{matrix} ]}^{\hat{} [ \begin{matrix}{P}_{1 , 1} & … & {P}_{1 , m} \\ … & … & … \\ {P}_{n , 1} & … & {P}_{n , m}\end{matrix} ]} ) \qquad ( Formula 14 )$$

Vascular conditions such as vasodilation, vasoconstriction, peripheral pressure decoupling, conditions where the peripheral arterial pressure is not proportional to the central aortic pressure, and conditions where the peripheral arterial pressure is lower than the central aortic pressure can be detected in a subject using χ and χₕ As a first example, the difference (Δχ) between χₕ and χ can be monitored.

[symbol]

χ=χₕ−χ (Formula 14)

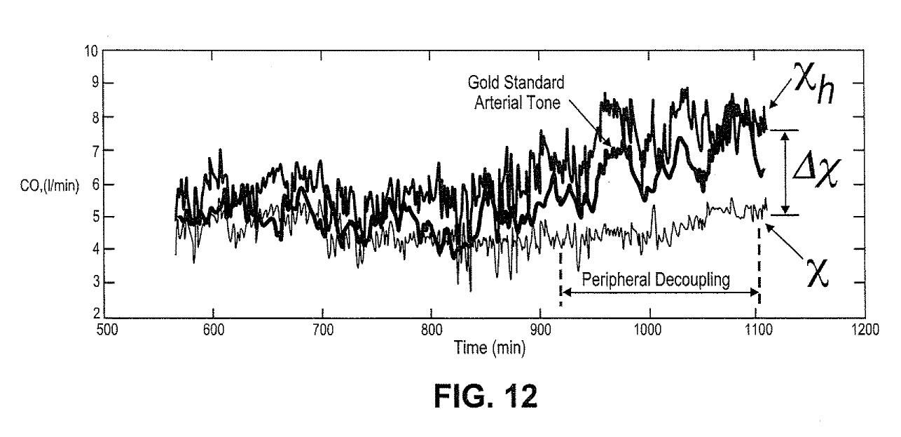

The difference between χ and χₕ indicates the vascular condition because χₕ uses the additional arterial pressure waveform parameters A_{sys}, t_{sys}, and t_{dia} that are sensitive to the vascular condition. Thus, an increasing Δχ shows changes in parameters A_{sys}, t_{sys}, and t_{dia} that indicate the vascular condition. This is because the model χₕ was approximated (during the numerical fitting) using combined data from patients in normal hemodynamic conditions and patients in extreme hyperdynamic conditions with peripheral decoupling, while the model χ was approximated using data only from patients with normal hemodynamic conditions. For this reason, the difference Δχ will be small for patients in normal conditions and it will be high for patients in hyperdynamic conditions when arterial tone is low and the peripheral pressure and flow are decoupled. FIG. 12 shows a calculation of χ (thin black line), χₕ (grey line) and gold standard arterial tone (thick black line) for a subject that entered hyperdynamic conditions at about the 950 minute mark of the provided time scale.

Another way to monitor vascular conditions in a subject is to calculate the ratio of χₕ to χ. When the ratio exceeds a predetermined value vasodilatory conditions are indicated. As an example, for the values of χₕ and χ shown in FIG. 12, the ratio of χₕ to χ increases after about the 950 minute mark of the provided time scale, i.e., the time after which the subject entered hyperdynamic conditions.

Other parameters based on the arterial tone factor such as, for example, Stroke Volume (SV), Cardiac Output (CO), Arterial Flow, or Arterial Elasticity can be used to monitor vascular conditions in a subject. As an example, Stroke Volume (SV) can be calculated as the product of the arterial tone and the standard deviation of the arterial pressure signal:

SV=χ·σ_{P} (Formula 15)

where:
SV is stroke volume;
χ is arterial tone; and
σₚ is the standard deviation of the arterial pressure

The difference in SV computed with the two different model could be used to detect the vascular condition, as follows:

ΔSV=(χₕ−χ)·σ_{P} (Formula 16)

### Measurement Interval

The analog measurement interval, that is, the time window [t0, tf], and thus the discrete sampling interval k=0, . . . , (n−1), over which each calculation period is conducted should be small enough so that it does not encompass substantial shifts in the pressure and/or time moments. However, a time window extending longer than one cardiac cycle will provide suitable data. Preferably, the measurement interval is a plurality of cardiac cycles that begin and end at the same point in different cardiac cycles. Using a plurality of cardiac cycles ensures that the mean pressure value used in the calculations of the various higher-order moments will use a mean pressure value P_{avg} that is not biased because of incomplete measurement of a cycle.

Larger sampling windows have the advantage that the effect of perturbations such as those caused by reflections are typically reduced. An appropriate time window can be determined using normal experimental and clinical methods well known to those of skill in the art. Note that it is possible for the time window to coincide with a single heart cycle, in which case mean pressure shifts will not be of concern.

The time window [t0, tf] is also adjustable according to drift in P_{avg}. For example, if P_{avg} over a given time window differs absolutely or proportionately by more than a threshold amount from the P_{avg} of the previous time window, then the time window can be reduced; in this case stability of P_{avg} is then used to indicate that the time window can be expanded. The time window can also be expanded and contracted based on noise sources, or on a measure of signal-to-noise ratio or variation. Limits are preferably placed on how much the time window is allowed to expand or contract and if such expansion or contraction is allowed at all, then an indication of the time interval is preferably displayed to the user.

The time window does not need to start at any particular point in the cardiac cycle. Thus, t₀ need not be the same as t_{dia0}, although this may be a convenient choice in many implementations. Thus, the beginning and end of each measurement interval (i.e., t0 and tf) may be triggered on almost any characteristic of the cardiac cycle, such as at times t_{dia0} or t_{sys}, or on non-pressure characteristics such as R waves, etc.

### Other Inputs

Rather than measure blood pressure directly, any other input signal may be used that is proportional to blood pressure. This means that calibration may be done at any or all of several points in the calculations. For example, if some signal other than arterial blood pressure itself is used as input, then it may be calibrated to blood pressure before its values are used to calculate the various component moments, or afterwards, in which case either the resulting moment values can be scaled. In short, the fact that the cardiovascular parameter may in some cases use a different input signal than a direct measurement of arterial blood pressure does not preclude its ability to generate an accurate compliance estimate.

### System Components

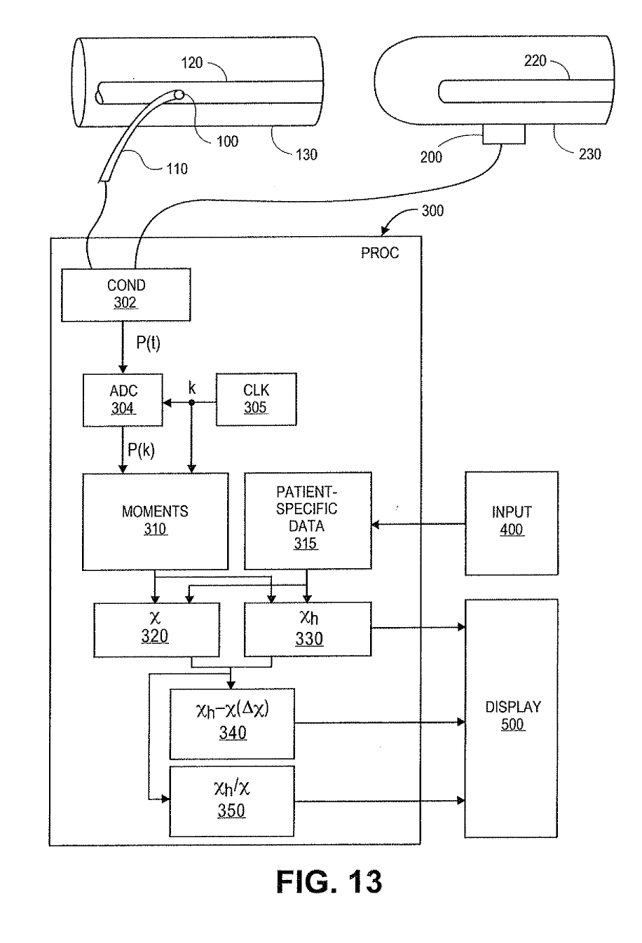

FIG. 13 shows the main components of a system that implements the methods described herein for detecting vascular conditions such as vasodilation in a subject. The methods may be implemented within an existing patient-monitoring device, or it may be implemented as a dedicated monitor. As is mentioned above, pressure, or some other input signal proportional to pressure, may be sensed in either or, indeed, both, of two ways: invasively and non-invasively. For convenience the system is described as measuring arterial blood pressure as opposed to some other input signal that is converted to pressure.

FIG. 13 shows both types of pressure sensing for the sake of completeness. In most practical applications of the methods described herein, either one or several variations will typically be implemented. In invasive applications of the methods described herein, a conventional pressure sensor 100 is mounted on a catheter 110, which is inserted in an artery 120 of a portion 130 of the body of a human or animal patient. The artery 120 is any artery in the arterial system, such as, for example, the femoral, radial or brachial artery. In the non-invasive applications of the methods described herein, a conventional pressure sensor 200, such as a photo-plethysmographic blood pressure probe, is mounted externally in any conventional manner, for example using a cuff around a finger 230 or a transducer mounted on the wrist of the patient. FIG. 13 schematically shows both types.

The signals from the sensors 100, 200 are passed via any known connectors as inputs to a processing system 300, which includes one or more processors and other supporting hardware and system software (not shown) usually included to process signals and execute code. The methods described herein may be implemented using a modified, standard, personal computer, or may be incorporated into a larger, specialized monitoring system. For use with the methods described herein, the processing system 300 also may include, or is connected to, conditioning circuitry 302 which performs normal signal processing tasks such as amplification, filtering, or ranging, as needed. The conditioned, sensed input pressure signal P(t) is then converted to digital form by a conventional analog-to-digital converter ADC 304, which has or takes its time reference from a clock circuit 305. As is well understood, the sampling frequency of the ADC 304 should be chosen with regard to the Nyquist criterion so as to avoid aliasing of the pressure signal (this procedure is very well known in the art of digital signal processing). The output from the ADC 304 will be the discrete pressure signal P(k), whose values may be stored in conventional memory circuitry (not shown).

The values P(k) are passed to or accessed from memory by a software module 310 comprising computer-executable code for computing whichever of the parameters μ_{1T} . . . μ_{kT}, μ_{1P} . . . μ_{kP}, etc. are to used in the chosen algorithm for calculating a cardiovascular parameter, such as χ and Xₕ. Even moderately skilled programmers will know how to design this software module 310.

The patient-specific data such as age, height, weight, BSA, etc., is stored in a memory region 315, which may also store other predetermined parameters such as Kₚᵣᵢₒᵣ. These values may be entered using any known input device 400 in the conventional manner.

Cardiovascular parameters χ and χₕ, are calculated by calculation modules 320 and 330. Calculation modules 320 and 330 include computer-executable code and take as inputs the various moment and patient-specific values, then performs the chosen calculations for computing χ and χₕ. For example, the modules 320 and 330 could enter the parameters into the expression given above for χ and χₕ, or into some other expression derived by creating an approximating function that best fits a set of test data. The calculation modules 320 and 330 preferably also select the time window [t0, tf] over which each χ and χₕ estimate is generated. This may be done as simply as choosing which and how many of the stored, consecutive, digitized P(t) values P(k) are used in each calculation, which is the same as selecting n in the range k=0, . . . , (n−1).

Further calculation modules 340 and 350 can be included to calculate Δχ and χₕ/χ as needed. The input to these calculation modules is from modules 320 and 330. The output of these modules is sent to the display 500 as desired.

As mentioned above, it is not necessary for the system according to the methods described herein to compute each of χ, χₕ, Δχ, and χₕ/χ if these values are not of interest. In such case, the corresponding software modules will of course not be needed and may be omitted. For example, the methods described herein could monitor only χₕ, in which case modules 320, 340, and 350 would not be needed. As illustrated by FIG. 13, any or all of the results χₕ, Δχ, and χₕ/χ may be passed to any conventional display or recording device 500 for presentation to and interpretation by a user. As with the input device 400, the display 500 will typically be the same as is used by the processing system for other purposes.

For each of the methods described herein, when the vascular condition is detected, a user can be notified of the vascular condition. The user can be notified of the vasodilatory conditions by publishing a notice on display 500 or another graphical user interface device. Further, a sound can be used to notify the user of the vascular condition. Both visual and auditory signals can be used.

Exemplary embodiments of the present invention have been described above with reference to a block diagram of methods, apparatuses, and computer program products. One of skill will understand that each block of the block diagram, and combinations of blocks in the block diagram, respectively, can be implemented by various means including computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the blocks.

The methods described herein further relate to computer program instructions that may be stored in a computer-readable memory that can direct a computer or other programmable data processing apparatus, such as in a processor or processing system (shown as 300 in FIG. 13), to function in a particular manner, such that the instructions stored in the computer-readable memory produce an article of manufacture including computer-readable instructions for implementing the function specified in the blocks illustrated in FIG. 13. The computer program instructions may also be loaded onto a computer, the processing system 300, or other programmable data processing apparatus to cause a series of operational steps to be performed on the computer, the processing system 300, or other programmable apparatus to produce a computer-implemented process such that the instructions that execute on the computer or other programmable apparatus provide steps for implementing the functions specified in the blocks. Moreover, the various software modules 320, 330, 340, and 350 used to perform the various calculations and perform related method steps described herein also can be stored as computer-executable instructions on a computer-readable medium in order to allow the methods to be loaded into and executed by different processing systems.

Accordingly, blocks of the block diagram support combinations of means for performing the specified functions, combinations of steps for performing the specified functions, and program instruction means for performing the specified functions. One of skill will understand that each block of the block diagram, and combinations of blocks in the block diagram, can be implemented by special purpose hardware-based computer systems that perform the specified functions or steps, or combinations of special purpose hardware and computer instructions.

The present invention is not limited in scope by the embodiments disclosed herein which are intended as illustrations of a few aspects of the invention and any embodiments which are functionally equivalent are within the scope of this invention. Various modifications of the apparatus and methods in addition to those shown and described herein will become apparent to those skilled in the art and are intended to fall within the scope of the appended claims. Further, while only certain representative combinations of the apparatus and method steps disclosed herein are specifically discussed in the embodiments above, other combinations of the apparatus components and method steps will become apparent to those skilled in the art and also are intended to fall within the scope of the appended claims. Thus a combination of components or steps may be explicitly mentioned herein; however, other combinations of components and steps are included, even though not explicitly stated. The term “comprising” and variations thereof as used herein is used synonymously with the term “including” and variations thereof and are open, non-limiting terms.

What is claimed is:

1. A method of detecting a vascular condition in a subject comprising:
receiving a signal corresponding to an arterial blood pressure;
calculating a cardiovascular parameter using the arterial blood pressure signal based on a set of factors including one or more parameters effected by the vascular condition; and
monitoring the cardiovascular parameter to determine if there is a statistically significant change over time,
wherein detection of the statistically significant change in the cardiovascular parameter indicates the vascular condition.

2. The method of claim 1, wherein the one or more parameters effected by the vascular condition are selected from the group consisting of (a) a parameter based on the area under the systolic portion of the arterial blood pressure signal, (b) a parameter based on the duration of systole, and (c) a parameter based on the ratio of the duration of the systole to the duration of the diastole.

3. The method of claim 2, wherein the one or more parameters effected by the vascular condition is a parameter based on the area under the systolic portion of the arterial blood pressure signal.

4. The method of claim 2, wherein the one or more parameters effected by the vascular condition is a parameter based on the duration of systole.

5. The method of claim 2, wherein the one or more parameters effected by the vascular condition is a parameter based on the ratio of the duration of the systole to the duration of the diastole.

6. The method of claim 2, wherein the one or more parameters effected by the vascular condition include (a) a parameter based on the area under the systolic portion of the arterial blood pressure signal, (b) a parameter based on the duration of systole, and (c) a parameter based on the ratio of the duration of the systole to the duration of the diastole.

7. The method of claim 1, further comprising calculating the cardiovascular parameter additionally using one or more of (d) a parameter based on the shape of the beat-to-beat arterial blood pressure signal and at least one statistical moment of the arterial blood pressure signal having an order of one or greater, (e) a parameter corresponding to the heart rate, and (f) a set of anthropometric parameters of the subject.

8. The method of claim 1, wherein the statistically significant change is a change of greater than one standard deviation.

9. The method of claim 1, wherein the cardiovascular parameter is arterial compliance.

10. The method of claim 1, wherein the cardiovascular parameter is arterial elasticity.

11. The method of claim 1, wherein the cardiovascular parameter is peripheral resistance.

12. The method of claim 1, wherein the cardiovascular parameter is arterial tone.

13. The method of claim 1, wherein the cardiovascular parameter is arterial flow.

14. The method of claim 1, wherein the cardiovascular parameter is stroke volume.

15. The method of claim 1, wherein the cardiovascular parameter is cardiac output.

16. The method of claim 1, wherein the vascular condition indicates the occurrence of vasodilation.

17. The method of claim 1, wherein the vascular condition indicates the occurrence of vasoconstriction.

18. The method of claim 1, wherein the vascular condition indicates the occurrence of hyperdynamic cardiovascular conditions.

19. The method of claim 1, wherein the presence of the vascular condition indicates hyperdynamic decoupling of the peripheral arterial pressure from the central aortic pressure.

20. The method of claim 1, wherein the presence of the vascular condition indicates that the peripheral arterial pressure is lower than the central aortic pressure.

21. The method of claim 1, wherein the presence of the vascular condition indicates that the peripheral arterial pressure is not proportional to the central aortic pressure.

22. The method of claim 1, wherein the cardiovascular parameter is calculated with an empirical multivariable statistical model using the following steps:
determining an approximating function relating a set of clinically derived reference measurements to the cardiovascular parameter, the approximating function being a function of at least (a) a parameter based on the area under the systolic portion of the arterial blood pressure signal, (b) a parameter based on the duration of systole, (c) a parameter based on the ratio of the duration of the systole to the duration of the diastole, and a set of clinically determined reference measurements of the cardiovascular parameter representing clinical measurements of the cardiovascular parameter from both subjects not experiencing the vascular condition and subjects experiencing the vascular condition;
determining a set of arterial blood pressure parameters from the arterial blood pressure signal, the set of arterial blood pressure parameters including at least (a) a parameter based on the area under the systolic portion of the arterial blood pressure signal, (b) a parameter based on the duration of systole, and (c) a parameter based on the ratio of the duration of the systole to the duration of the diastole;
estimating the cardiovascular parameter by evaluating the approximating function with the set of arterial blood pressure parameters.

23. The method of claim 22, further comprising determining the approximating function additionally using one or more of (d) a parameter based on the shape of the arterial blood pressure signal and at least one statistical moment of the arterial blood pressure signal having an order of one or greater, (e) a parameter corresponding to the heart rate, and (f) a set of anthropometric parameters of the subject.

24. The method of claim 22, wherein the data derived from subjects experiencing the vascular condition is given more weight in the model than the data derived from subjects not experiencing the vascular condition.

25. The method of claim 1, further comprising alerting a user when the vascular condition is detected.

26. The method of claim 25, wherein the user is alerted by publishing a notice on a graphical user interface.

27. The method of claim 25, wherein a user is alerted by emitting a sound.

28. A method of detecting a vascular condition in a subject comprising:
receiving a signal corresponding to an arterial blood pressure;
calculating a first cardiovascular parameter using the arterial blood pressure signal based on a first set of factors including one or more of (a) a parameter based on the shape of the beat-to-beat arterial blood pressure signal and at least one statistical moment of the arterial blood pressure signal having an order of one or greater, (b) a parameter based on a heart rate of the subject, and (c) a set of anthropomorphic parameters of the subject;
calculating a second cardiovascular parameter using the arterial blood pressure signal based on the first set of factors and one or more parameters effected by the vascular condition; and
calculating a difference factor by subtracting the first cardiovascular parameter from the second cardiovascular parameter,
wherein the difference factor being greater than a predetermined threshold value indicates the vascular condition.

29. The method of claim 28, wherein the one or more parameters effected by the vascular condition are selected from the group consisting of (a) a parameter based on the area under the systolic portion of the arterial blood pressure signal, (b) a parameter based on the duration of systole, and (c) a parameter based on the ratio of the duration of the systole to the duration of the diastole.

30. The method of claim 29, wherein the one or more parameters effected by the vascular condition is a parameter based on the area under the systolic portion of the arterial blood pressure signal.

31. The method of claim 29, wherein the one or more parameters effected by the vascular condition is a parameter based on the duration of systole.

32. The method of claim 29, wherein the one or more parameters effected by the vascular condition is a parameter based on the ratio of the duration of the systole to the duration of the diastole.

33. The method of claim 29, wherein the one or more parameters effected by the vascular condition include (a) a parameter based on the area under the systolic portion of the arterial blood pressure signal, (b) a parameter based on the duration of systole, and (c) a parameter based on the ratio of the duration of the systole to the duration of the diastole.

34. The method of claim 28, wherein the predetermined threshold value is a statistically significant change in the difference factor over time.

35. The method of claim 28, wherein the predetermined threshold value is about 1.5 L/minute or greater.

36. The method of claim 28, wherein the predetermined threshold value is about 1.8 L/minute or greater.

37. The method of claim 28, wherein the predetermined threshold value is about 2 L/minute.

38. The method of claim 28, wherein the predetermined threshold value is about 2.5 L/minute or greater.

39. The method of claim 28, wherein the cardiovascular parameter is arterial compliance.

40. The method of claim 28, wherein the cardiovascular parameter is arterial elasticity.

41. The method of claim 28, wherein the cardiovascular parameter is peripheral resistance.

42. The method of claim 28, wherein the cardiovascular parameter is arterial tone.

43. The method of claim 28, wherein the cardiovascular parameter is arterial flow.

44. The method of claim 28, wherein the cardiovascular parameter is stroke volume.

45. The method of claim 28, wherein the cardiovascular parameter is cardiac output.

46. The method of claim 28, wherein the vascular condition indicates the occurrence of vasodilation.

47. The method of claim 28, wherein the vascular condition indicates the occurrence of vasoconstriction.

48. The method of claim 28, wherein the vascular condition indicates the occurrence of hyperdynamic cardiovascular conditions.

49. The method of claim 28, wherein the vascular condition indicates hyperdynamic decoupling of the peripheral arterial pressure from the central aortic pressure.

50. The method of claim 28, wherein the vascular condition indicates that the peripheral arterial pressure is lower than the central aortic pressure.

51. The method of claim 28, wherein the vascular condition indicates that the peripheral arterial pressure is not proportional to the central aortic pressure.

52. The method of claim 28, wherein the first cardiovascular parameter is calculated with an empirical multivariable statistical model using the following steps:
determining an approximating function relating a set of clinically derived reference measurements to the first cardiovascular parameter, the approximating function being a function of at least (a) a parameter based on the shape of the arterial blood pressure signal including calculating at least one statistical moment of the arterial blood pressure signal having an order of one or higher, (b) a parameter based on the heart rate, and (c) a set of anthropometric parameters of the subject, and the set of clinically determined reference measurements of the first cardiovascular parameter representing clinical measurements of the first cardiovascular parameter from subjects not experiencing the vascular condition;
determining a set of arterial blood pressure parameters from the arterial blood pressure signal, the set of arterial blood pressure parameters including at least the shape of the arterial blood pressure signal and at least one statistical moment of the arterial blood pressure signal having an order of one or greater, and the heart rate;
determining a set of anthropometric parameters of the subject; and
estimating the first cardiovascular parameter by evaluating the approximating function with the set of arterial blood pressure parameters and the set of anthropometric parameters of the subject.

53. The method of claim 28, wherein the second cardiovascular parameter is calculated with an empirical multivariable statistical model using the following steps:
determining an approximating function relating a set of clinically derived reference measurements to the first cardiovascular parameter, the approximating function being a function of at least (a) the parameter or parameters used to calculate the first cardiovascular function, (b) a parameter based on the area under the systolic portion of the arterial blood pressure signal, (c) a parameter based on the duration of systole, and (d) a parameter based on the ratio of the duration of the systole to the duration of the diastole, and the set of clinically determined reference measurements of the second cardiovascular parameter representing clinical measurements of the second cardiovascular parameter from both subjects not experiencing the vascular condition and subjects experiencing the vascular condition;
determining a set of arterial blood pressure parameters from the arterial blood pressure signal, the set of arterial blood pressure parameters including at least (a) the parameter or parameters used to calculate the first cardiovascular function, (b) a parameter based on the area under the systolic portion of the arterial blood pressure signal, (c) a parameter based on the duration of systole, and (d) a parameter based on the ratio of the duration of the systole to the duration of the diastole;
estimating the second cardiovascular parameter by evaluating the approximating function with the set of arterial blood pressure parameters.

54. The method of claim 53, wherein the data derived from subjects experiencing the vascular condition is given more weight in the model than the data derived from subjects not experiencing the vascular condition.

55. The method of claim 28, further comprising alerting a user when the vascular condition is detected.

56. The method of claim 55, wherein the user is alerted by publishing a notice on a graphical user interface.

57. The method of claim 55, wherein a user is alerted by emitting a sound.

58. A method of detecting a vascular condition in a subject comprising:
receiving a signal corresponding to an arterial blood pressure;
calculating a first cardiovascular parameter using the arterial blood pressure signal based on a first set of factors including one or more of (a) a parameter based on the shape of the arterial blood pressure signal and at least one statistical moment of the arterial blood pressure signal having an order of one or greater, (b) a parameter based on a heart rate of the subject, and (c) a set of anthropomorphic parameters of the subject; and
calculating a second cardiovascular parameter using the arterial blood pressure signal based on the first set of factors and one or more parameters effected by the vascular condition;
wherein a ratio of the second cardiovascular parameter to the first cardiovascular parameter greater than a predetermined value indicates the vascular condition.

59. The method of claim 58, wherein the one or more parameters effected by the vascular condition are selected from the group consisting of (a) a parameter based on the area under the systolic portion of the arterial blood pressure signal, (b) a parameter based on the duration of systole, and (c) a parameter based on the ratio of the duration of the systole to the duration of the diastole.

60. The method of claim 59, wherein the one or more parameters effected by the vascular condition is a parameter based on the area under the systolic portion of the arterial blood pressure signal.

61. The method of claim 59, wherein the one or more parameters effected by the vascular condition is a parameter based on the duration of systole.

62. The method of claim 59, wherein the one or more parameters effected by the vascular condition is a parameter based on the ratio of the duration of the systole to the duration of the diastole.

63. The method of claim 59, wherein the one or more parameters effected by the vascular condition include (a) a parameter based on the area under the systolic portion of the arterial blood pressure signal, (b) a parameter based on the duration of systole, and (c) a parameter based on the ratio of the duration of the systole to the duration of the diastole.

64. The method of claim 58, wherein the predetermined value is about 1.2 or greater.

65. The method of claim 58, wherein the predetermined value is about 1.3 or greater.

66. The method of claim 58, wherein the predetermined value is about 1.4 or greater.

67. The method of claim 58, wherein the predetermined value is about 1.5 or greater.

68. The method of claim 58, wherein the cardiovascular parameter is arterial compliance.

69. The method of claim 58, wherein the cardiovascular parameter is arterial elasticity.

70. The method of claim 58, wherein the cardiovascular parameter is peripheral resistance.

71. The method of claim 58, wherein the cardiovascular parameter is arterial tone.

72. The method of claim 58, wherein the cardiovascular parameter is arterial flow.

73. The method of claim 58, wherein the cardiovascular parameter is stroke volume.

74. The method of claim 58, wherein the cardiovascular parameter is cardiac output.

75. The method of claim 58, wherein the vascular condition indicates the occurrence of vasodilation.

76. The method of claim 58, wherein the vascular condition indicates the occurrence of vasoconstriction.

77. The method of claim 58, wherein the vascular condition indicates the occurrence of hyperdynamic cardiovascular conditions.

78. The method of claim 58, wherein the vascular condition indicates hyperdynamic decoupling of the peripheral arterial pressure from the central aortic pressure.

79. The method of claim 58, wherein the vascular condition indicates that the peripheral arterial pressure is lower than the central aortic pressure.

80. The method of claim 58, wherein the vascular condition indicates that the peripheral arterial pressure is not proportional to the central aortic pressure.

81. The method of claim 58, wherein the first cardiovascular parameter is calculated with an empirical multivariable statistical model using the following steps:
determining an approximating function relating a set of clinically derived reference measurements to the first cardiovascular parameter, the approximating function being a function of at least (a) a parameter based on the shape of the arterial blood pressure signal including calculating at least one statistical moment of the arterial blood pressure signal having an order of one or higher, (b) a parameter based on the heart rate, and (c) a set of anthropometric parameters of the subject, and the set of clinically determined reference measurements of the first cardiovascular parameter representing clinical measurements of the first cardiovascular parameter from subjects not experiencing the vascular condition;
determining a set of arterial blood pressure parameters from the arterial blood pressure signal, the set of arterial blood pressure parameters including at least the shape of the arterial blood pressure signal and at least one statistical moment of the arterial blood pressure signal having an order of one or greater, and the heart rate;
determining a set of anthropometric parameters of the subject; and
estimating the first cardiovascular parameter by evaluating the approximating function with the set of arterial blood pressure parameters and the set of anthropometric parameters of the subject.

82. The method of claim 58, wherein the second cardiovascular parameter is calculated with an empirical multivariable statistical model using the following steps:
determining an approximating function relating a set of clinically derived reference measurements to the first cardiovascular parameter, the approximating function being a function of at least (a) the parameter or parameters used to calculate the first cardiovascular function, (b) a parameter based on the area under the systolic portion of the arterial blood pressure signal, (c) a parameter based on the duration of systole, and (d) a parameter based on the ratio of the duration of the systole to the duration of the diastole, and the set of clinically determined reference measurements of the second cardiovascular parameter representing clinical measurements of the second cardiovascular parameter from both subjects not experiencing the vascular condition and subjects experiencing the vascular condition;
determining a set of arterial blood pressure parameters from the arterial blood pressure signal, the set of arterial blood pressure parameters including at least (a) the parameter or parameters used to calculate the first cardiovascular function, (b) a parameter based on the area under the systolic portion of the arterial blood pressure signal, (c) a parameter based on the duration of systole, and (d) a parameter based on the ratio of the duration of the systole to the duration of the diastole;
estimating the second cardiovascular parameter by evaluating the approximating function with the set of arterial blood pressure parameters.

83. The method of claim 82, wherein the data derived from subjects experiencing the vascular condition is given more weight in the model than the data derived from subjects not experiencing the vascular condition.

84. The method of claim 58, further comprising alerting a user when the vascular condition is detected.

85. The method of claim 84, wherein the user is alerted by publishing a notice on a graphical user interface.

86. The method of claim 84, wherein a user is alerted by emitting a sound.
